(19) United States
(12) Patent Application Publication
(10) Pub. No.: US 2003/0228598 A1
(43) Pub. Date: Dec. 11, 2003

(54) DNA/RNA as a write/read medium

(75) Inventor: Raymond P. Mariella JR., Danville, CA (US)

Correspondence Address: Eddie E. Scott, Assistant Laboratory Counsel, Lawrence Livermore National Laboratory, P.O. Box 808, L-703, Livermore, CA, 94551, US

(73) Assignee: The Regents of the University of California

(21) Appl. No.: 10/393,637

(22) Filed: Mar. 19, 2003

Related U.S. Application Data
(60) Provisional application No. 60/367,988, filed on Mar. 25, 2002.

(51) Int. Cl. C12Q 1/68; G06F 19/00; G01N 33/48; G01N 33/50

(52) U.S. Cl. 435/6; 702/20

(57) ABSTRACT

A system for writing and/or reading information using DNA. The information is translated into at least one information containing DNA sequence. At least one basic DNA sequence is preselected. A DNA molecule of user-defined sequence that contains said at least one information containing DNA sequence and said at least one basic DNA sequence is synthesized.

### CROSS-REFERENCE TO RELATED APPLICATIONS

This application claims the benefit of U.S. Provisional Patent Application No. 60/367,988 filed Mar. 25, 2002 titled “DNA/RNA as a Write/Read Medium.” U.S. Provisional Patent Application No. 60/367,988 filed Mar. 25, 2002 titled “DNA/RNA as a Write/Read Medium” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to DNA and RNA and more particularly to DNA and RNA as a write, read, and write and read medium.

2. State of Technology

U.S. Pat. No. 5,139,812 issued Aug. 18, 1992 describes a system for high security crypto-marking for protecting valuable objects. The system uses nucleic acid fragments which are specified by their sequence, their size, and their nature, and which are suitable for being used as detection targets in valuable objects such as works of art, durable goods, official papers, contracts, etc. A target nucleic acid can easily be hidden for subsequent detection, thereby providing proof of the ownership or the authenticity of a valuable object. The detection may be direct or by hybridization.

U.S. Pat. No. 6,167,518 issued Dec. 26, 2001 to Padgett et al. describes a system for forming a digital certificate representation of a unique biological feature of a registrant such as the registrant's chromosomal DNA. A document and the certificate are transmitted to a receiving terminal. The identity of the transmitting party can be verified by inspecting the certificate. In the event the sending party denies sending the document, the biological feature can be extracted from the certificate and directly compared with the actual biological feature of the sending party.

U.S. Pat. No. 6,312,911 issued Nov. 6, 2001 to Bancroft et al. describes DNA-based Steganography. A DNA encoded message is concealed within a genomic DNA sample followed by further concealment of the DNA sample to in microdot.

International Patent Application WO 02/095073 by Peter J. Belshaw et al. published Nov. 28, 2002 for a method for the synthesis of DNA sequences provides the following background information, “Using the techniques of recombinant DNA chemistry, it is now common for DNA sequences to be replicated and amplified from nature and for those sequences to then be disassembled into component parts which are then recombined or reassembled into new DNA sequences. While it is now both possible and common for short DNA sequences, referred to as oligonucleotides, to be directly synthesized from individual nucleosides, it has been thought to be generally impractical to directly construct large segments or assemblies of DNA sequences larger than about 400 base pairs. As a consequence, larger segments of DNA are generally constructed from component parts and segments which can be purchased, cloned or synthesized individually and then assembled into the DNA molecule desired.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for writing and/or reading information using DNA. The information is translated into at least one information containing DNA sequence. At least one basic DNA sequence is preselected. A DNA molecule of user-defined sequence that contains said at least one information containing DNA sequence and said at least one basic DNA sequence is synthesized.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates a system for writing DNA.

FIG. 2 illustrates one embodiment of a system for synthesizing a DNA molecule with information that is desired to be written into the DNA molecule.

FIG. 3 illustrates another embodiment of a system for synthesizing a DNA molecule with information that is desired to be written into the DNA molecule.

FIG. 4 illustrates the beginning of the synthesis of a DNA molecule with a surface-tethered, pre-defined, double-stranded, sequences of DNA approximately 30 base pairs long with a short, single-stranded overhang.

FIG. 5 illustrates an oligo of six bases used as the user-selected, single stranded DNA sequence.

FIG. 6 illustrates the selected oligo annealing to the initial DNA sequence by way of hydrogen bonding to the overhanging strand, thereby generating a new overhang.

FIG. 7 illustrates process being repeated with additional oligos until the desired full-length DNA sequence has been constructed.

FIG. 8 illustrates use of a pre-defined double-stranded sequence of approximately 30 base pairs in length to finish the DNA sequence.

FIG. 9 illustrates the final full-length DNA product.

### DETAILED DESCRIPTION OF THE INVENTION

Referring now to the drawings, to the following detailed description, and to incorporated materials; detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The present invention provides a system for writing DNA and/or RNA, reading DNA and/or RNA, and writing and reading DNA and/or RNA. The system allows information to be written in the medium of DNA and/or RNA. Uses of the system include attaching specific information to DNA and/or RNA. For example, information may be stored in DNA and/or RNA. Another example is the use of DNA to transmit encoded messages. Another example is the identification of an animal by providing identification information in the animal's DNA. Another example is information about a plant or animal may be written into the plant's or animal's DNA.

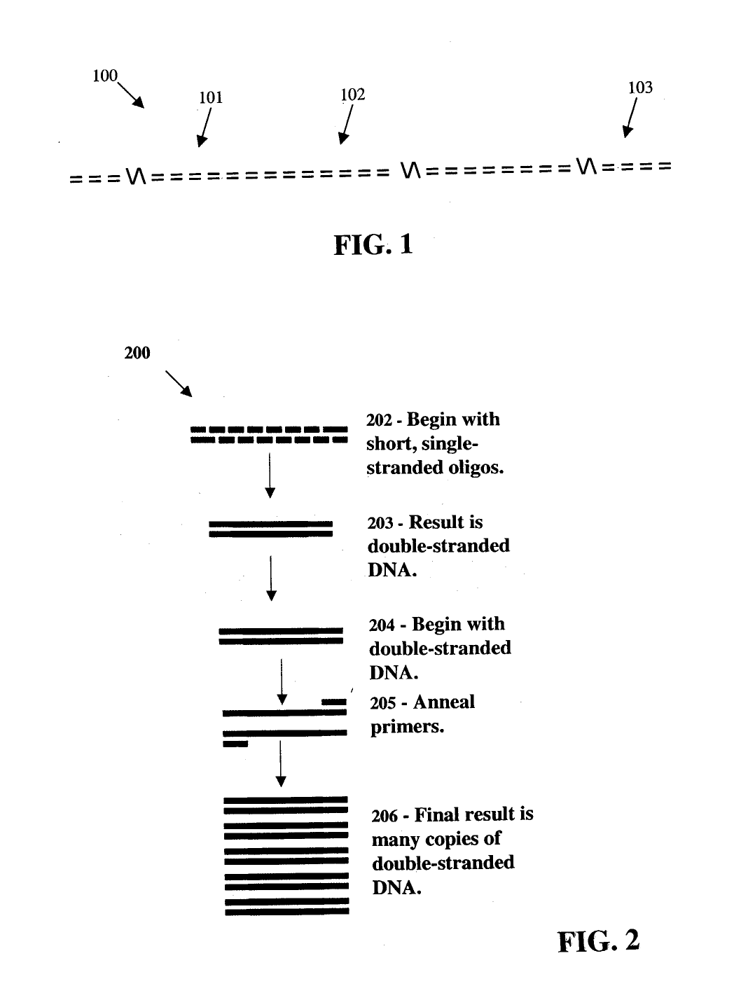

Referring now to FIG. 1, a system for writing DNA is illustrated. A long DNA molecule is designated generally by the reference numeral 100. The DNA molecule 100 is created by synthesis. There are different methods of synthesizing the DNA molecule 100. For example, the DNA molecule can be synthesized using array technology that is known in the art. For example, U.S. Pat. No. 6,238,868, incorporated herein by reference, provides the following information, “microchip device is an electronically controlled microelectrode array. See, PCT application WO96/01836, the disclosure of which is hereby incorporated by reference. In contrast to the passive hybridization environment of most other microchip devices, the electronic microchip devices (or active microarray devices) of the present invention offer the ability to actively transport or electronically address nucleic acids to discrete locations on the surface of the microelectrode array, and to bind the addressed nucleic acid at those locations to either the surface of the microchip at specified locations.” Another method of synthesizing the DNA molecule 100 is shown in International Patent Application WO 02/095073 by Peter J. Belshaw et al. for a method for the synthesis of DNA sequences published Nov. 28, 2002, incorporated herein by reference. Other methods of synthesizing the DNA molecule 100 will be described subsequently.

Once the specific DNA molecule 100 that is to be synthesized has been determined, the DNA molecule is broken into segments by a computer program. The segments combined and assembled to produce the DNA molecule 100 in accordance with the present invention. The DNA molecule 100 includes portions 101 and 103 constructed according to the sequence of the specific DNA molecule that is being synthesized. The DNA molecule 100 also includes a portion 102 constructed so that it contains the information that is being written into the DNA molecule 100.

There are different methods for translating the information that is being written into the DNA molecule 100 into the sequence units for the portion 102. U.S. Pat. No. 6,312,911, incorporated herein by reference, provides an example wherein a simple three-base code to represent each letter of the alphabet may be used; e.g., the three-base sequences AAA, AAC, AAG, and CCC might represent, respectively, the alphabet letters A, B, C and D. Another method for translating the information that is being written into the DNA molecule 100 into the sequence units for the portion 102 is a system called “Gencryption.” In Gencryption, the message is similar to a protein sequence and the encoded or encrypted message is similar to a DNA sequence. Decoding in Gencryption does have some similarities to transcription and translation in Biology. Each letter is converted to a three letter codon consisting of the four letters A, G, T, and C. Conversion tables are used to code and decode the message.

Referring now to FIG. 2, an embodiment is illustrated that includes a system for synthesizing a DNA molecule with the information that is desired written into the DNA molecule. The system is designated generally by the reference numeral 200. A desired sequence is pre-selected. The pre-selected sequence includes the information to be included in the DNA.

The system 200 begins by using computational techniques to break the desired sequence into fragments of defined size. These base fragments are then arrayed in groups and assembled into double-strand DNA molecules using DNA polymerase synthesis. As illustrated in FIG. 2, the polymerase-based synthesis system 200 begins with short, single-stranded oligos 202. The double-strand DNA molecules include the information to be included in the DNA. The products of these reactions are then combined, in as many steps as necessary, and assembled by polymerase into still-longer molecules, until the final desired product is assembled. The final product is then be amplified using PCR. This results in double-stranded DNA 203. The next step begins with doubled stranded DNA 204. The next step is to anneal primers 205. The final result is many copies of double-stranded DNA 206. The final product 206 includes the information to be included in the DNA.

In other embodiments of the present invention different systems for producing the DNA or RNA are used. In another embodiment a system for making very long, double-stranded synthetic polynucleotides is used. This system comprises sequentially hybridizing short single-stranded oligonucleotides (oligos) to each other, followed by enzymatic ligation. This results in a contiguous piece of PCR-ready double-stranded DNA of predetermined sequence that can be extended many thousands of base pairs. Caches of the different possible DNA hexamers are synthesized by conventional phosphoramidite synthesis prior to the long poly-nucleotide synthesis, and kept in the synthesis device to be drawn upon as need to create the desired molecule. This makes the long-strand nucleotide synthesis independent of in loco phosphoramidite syntheses. Since phosphoramidite synthesis is a fairly slow process requiring expensive and bulky equipment, the ability to pre-synthesize all of the components results in a significantly streamlined process. This procedure can be used to synthesize artificial genes, DNA or RNA probes, primers or any other molecule made of ribonucleic or deoxyribonucleic acid.

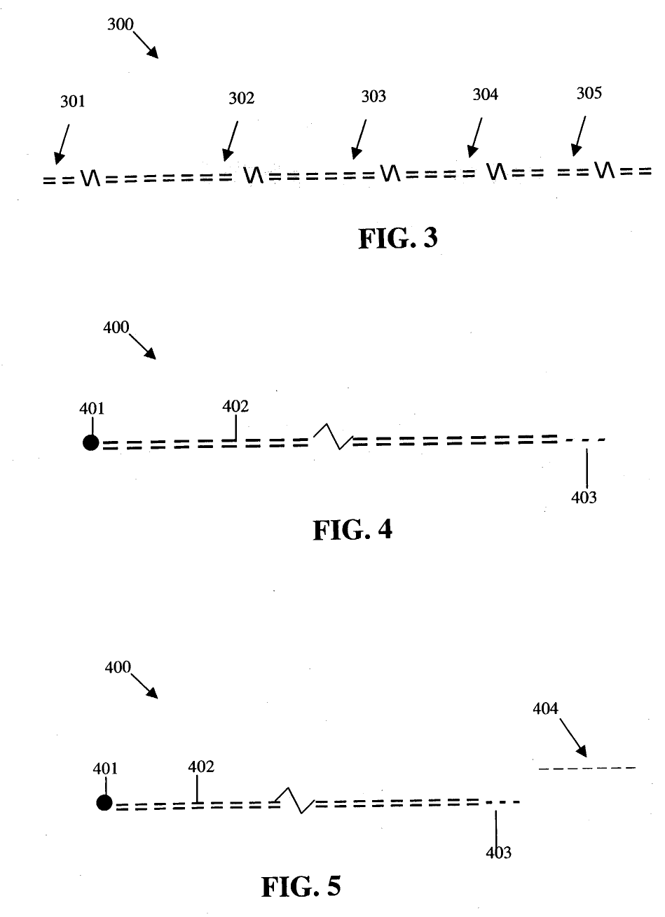

It becomes important to know where the information (message) begins in the DNA sequence. FIG. 3 illustrates another embodiment of a system for synthesizing a DNA molecule with information that is desired to be written into the DNA molecule. A long DNA molecule is designated generally by the reference numeral 300. The DNA molecule 300 is created by synthesis as previously described. The DNA molecule 300 includes portions 301 and 305 constructed according to the sequence of the specific DNA molecule that is being synthesized. The DNA molecule 300 also includes a portion 303 constructed so that it contains the information that is being written into the DNA molecule 300. A pre-defined string of DNA bases 302 and 304 are located before and after the portion 303 that contains the information that is being written into the DNA molecule.

There are be two basic approaches—one would always use exactly the same number (e.g. 100) of DNA bases as a parsable “line” of text. An alternative is to permit variable lengths of the parsable line. The alternative method necessitates limiting the base sequences that could be incorporated in the “text,” since a particular sequence would have to be reserved as a “stop-reading” sequence to signify the end of the variable-format line of text. Therefore, Applicant's first embodiment uses the fixed-line-length approach. One needs to reserve only the base sequences that uniquely identified the line of text (i.e. signified the line numbers.

Another embodiment of Applicant's invention comprises writing a message in a series of parsable “lines.” The system includes a DNA sequencer having single-base resolution. The sequencer can reliably deliver single-base resolution for the desired length of message lines. Physically large sequencing instruments are currently available that can read 800 to 1000 bases at the desired performance. There are also small instruments, based on plastic channels, that can work reliably out to 100 base-length reads.

The system can be better understood by considering the following example: (1) Let us use “N” to represent the sequence read length for which the instrument reliably delivered accurate, single-base resolution. (2) Using the analogy of lines of text on a printed page, one would write the equivalent of lines of text in the DNA sequence, each line of which would begin with the equivalent of a carriage return/line-feed character as a symbolic line deliminator. (3) Each line would be N DNA bases long. (4) The key difference is that lines of text on a printed page have spatial separations that are easy for the human eye to see, so that all lines may be deliminated by the same beginning-of-line character. (5) For the DNA writing and reading, a unique line deliminator is needed for each parsed line of DNA “text.”

The system described above uses a simple version using AAAAT7fiIT as the equivalent of the carriage-return character, immediately followed by a concatenated series of (AT) pairs as the unique line-feed characters with the number of pairs “k” identifying which line is being terminated. Given that Applicant desires to use Sanger-style polymerase-based chemical reactions (SPC) preparatory to reading out the lines, using the AAAAATTTTT(AT)k as unique line deliminator is highly unappealing; particularly if one needs 100 lines or more or text.

Applicant will now describe another way to write the unique line deliminators. Once again, a common “carriage-return character” in the DNA, such as AAAAATTIT= is used, but this is immediately followed by a specified number of DNA bases whose internal composition will uniquely label the number of the line of text. A trivial, binary analogy would be to use an 8-base sequence as the unique line deliminator.

For example, use A to represent “0” and T to represent “1” in a binary number. Thus, AAAAAAAA represents zero, AAAAAAAT represents one, AAAAAATA represents two, AAAAATAA represents four, AAAATTAT represents thirteen, etc. This 8-basesequence binary approach would allow the unique labeling of 256 (28) parsable lines of DNA “text.” Assuming an N-base-long line of DNA text, he entire message could, thus, be 256*N bases long, ignoring the technical difficulties of synthesizing and maintaining this sequence. Thus, each SPC primer for the DNA sequencer to read would have the complement of the “carriage return concatenated with the complement (A-T and G-C complementary [WatsonCrick] base pairing in the double helix) of the unique line deliminator. That is, the primer for the “zeroth” line of DNA text would be TI′TTTTTTAAAAATTTTT. Each parsable line of DNA text would have its unique 18-base-long SPC primer, in this example. If one desired to shorten the line deliminators and corresponding SPC primers, one could employ three or four DNA bases for the unique identifiers. If one used three bases, a 5-base-long identifier could uniquely label 64 (35) parsable lines of DNA text. Similarly, if one used all four DNA bases, a 4-base-long identifier could uniquely label 256 (44) parsable lines of DNA text.

There are some problems associated with using three or four DNA bases, in that organisms recognize certain sequences, such as “ATG,” as genetic instructions. If these sequences were never present within a living organism, this would not be a problem. If these DNA sequences were ever inserted within the cellular machinery that reads and acts upon DNA sequences, then the sequence ATG may need to be skipped, reducing the total number of usable unique line labels.

Referring now to FIGS. 4-9 of the drawings, another embodiment of a system for synthesizing a DNA molecule with the information that is desired written into the DNA molecule is illustrated. The DNA molecule is designated generally by the reference numeral 400. Once the specific DNA molecule 400 that is to be synthesized has been determined, the DNA molecule is broken into segments by a computer program. The segments combined and assembled to produce the DNA molecule 400 in accordance with the present invention. The DNA molecule 400 includes portions constructed according to the sequence of the specific DNA molecule that is being synthesized. The DNA molecule 400 also includes a portion constructed so that it contains the information that is being written into the DNA molecule 400.

As illustrated in FIG., 4, the synthesis of the DNA molecule 400 begins with surface-tethered, pre-defined, double-stranded, sequences of DNA approximately 30 base pairs long with a short, single-stranded overhang. The surface-tethered, pre-defined, double-stranded, sequences of DNA is a T7 primer 402. This type of primer is commercially available. The T7 primer 402 has a short, single-stranded overhang 403. The overhang 403 comprises a three bases overhang. A bead 401 is attached to the T7 primer 402. The surface is voltage controlled according to systems known in the art.

Construction of the full-length DNA product involves a repetitive process in which the initial DNA sequence is lengthened by the addition of a user-selected, single stranded DNA sequence, called an oligonucleotide (“oligo”) comprised of approximately 6 (or more) bases. As illustrated by FIG. 5, an oligo 404 of six bases is used as the user-selected, single stranded DNA sequence. The oligo 404 and subsequent oligos contain the information that is being written into the DNA molecule 400. As explained above there are different methods for translating the information that is being written into the sequence units of oligo 404 and subsequent oligos.

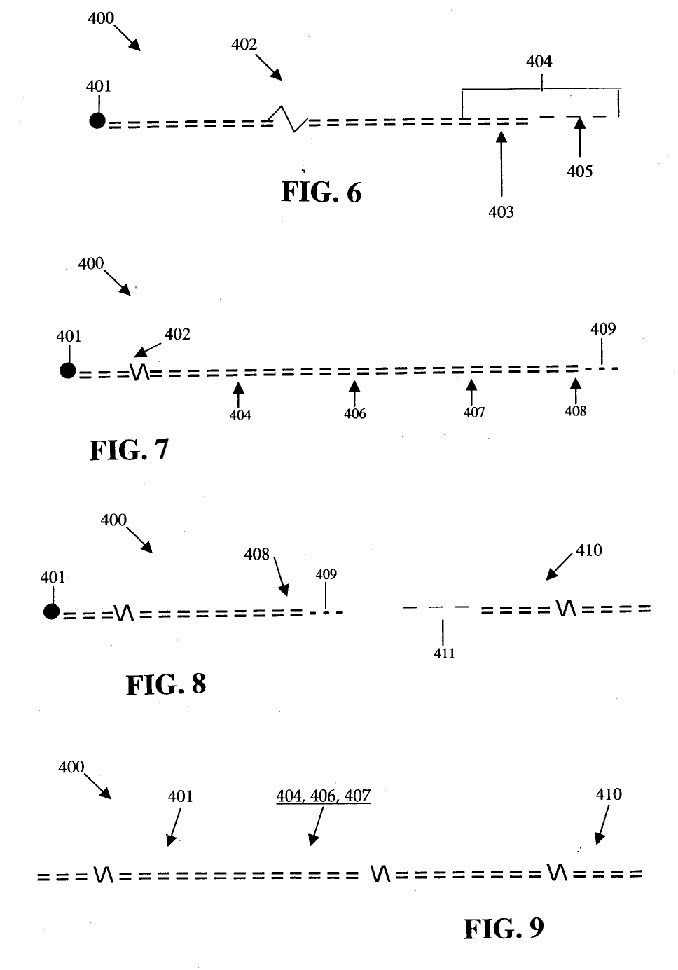

The selected oligo 404 anneals to the initial DNA sequence by way of hydrogen bonding to the overhanging strand, thereby generating a new overhang. The bases at the proximal end of the oligo 404 must, therefore, be complementary to the overhanging bases. The oligo 404 is then covalently attached to the initial sequence using an enzyme called ligase. As illustrated by FIG. 6, the selected oligo 404 anneals to the initial DNA sequence 402 by way of hydrogen bonding to the overhanging strand 403, thereby generating a new overhang 405. The three bases at the proximal end of the oligo 404 must are complementary to the overhanging three bases 403 on the T7 primer 402. The oligo 404 is then covalently attached to the initial sequence using an enzyme called ligase.

The excess oligo and ligase are removed, and the process is repeated with additional oligos until the desired full-length DNA sequence has been constructed. The oligo 404 and subsequent oligos contain the information that is being written into the DNA molecule 400. As illustrated by FIG. 7, the excess oligo and ligase are removed. The process is repeated with additional oligos 406, 407, 408, etc. until the desired full-length DNA molecule 400 has been constructed. The DNA molecule contains the information written into the DNA molecule 400. The oligos 404, 406, 407, etc. contain the information written into the DNA molecule 400.

After the last oligo 408 has been attached, the DNA sequence is finished by ligating a pre-defined double-stranded sequence approximately 30 base pairs in length, which has a single-stranded overhang complementary to the overhang of the final oligo. This 30-base pair sequence may either be identical to or different than the first sequence that was attached to the surface.

As illustrated by FIG. 8 a pre-defined double-stranded sequence 410 approximately 30 base pairs in length is used to finish the DNA sequence 400. The sequence 410 has a single-stranded overhang complementary to the overhang 409 of the final oligo 408. The final step involves PCR amplification of the full-length sequence 400 using primers complementary to the 30-base pair termini. The final full-length DNA product 400 is illustrated in FIG. 9. The full-length DNA product 400 comprises T7 primer 402, the oligos 404, 406, 407 etc. containing the information written into the DNA molecule and the pre-defined double-stranded sequence 410.

The detailed description, incorporated materials, drawings, and claims provide information about the invention. The information serves to explain the principles of the invention. The invention is susceptible to various modifications and alternative forms. It is to be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of writing and/or reading information using DNA, comprising the steps of:
translating said information into at least one information containing DNA sequence,
preselecting at least one basic DNA sequence,
synthesizing a DNA molecule of user-defined sequence that contains said at least one information containing DNA sequence and said at least one basic DNA sequence.

2. The method of writing and/or reading information using DNA of claim 1 wherein said step of translating said information into at least one information containing DNA sequence comprises using a computer program to translate said information into at least one information containing DNA sequence.

3. The method of writing and/or reading information using DNA of claim 1 wherein said step of preselecting at least one basic DNA sequence comprises using a computer program to preselect at least one basic DNA sequence.

4. The method of writing and/or reading information using DNA of claim 1 wherein said steps of translating said information into at least one information containing DNA sequence and preselecting at least one basic DNA sequence compris using computational techniques to break said sequences into fragments of defined size and said step of synthesizing a DNA molecule of user-defined sequence comprises assembling said fragments into said DNA molecule of user-defined sequence.

5. The method of writing and/or reading information using DNA of claim 1 wherein pre-defined strings of DNA bases are located before and after said at least one information containing DNA sequence in said DNA molecule of user-defined sequence.

6. The method of writing and/or reading information using DNA of claim 1 wherein said steps of synthesizing a DNA molecule of user-defined sequence comprises providing a pre-defined, double-stranded, sequence of DNA with a single-stranded overhang, tethered said pre-defined, double-stranded, sequence of DNA with a single-stranded overhang, and lengthening said pre-defined, double-stranded, sequences of DNA by the addition of user-selected, single stranded DNA sequences.

7. The method of writing and/or reading information using DNA of claim 1 wherein said steps of synthesizing a DNA molecule of user-defined sequence comprises providing a pre-defined, double-stranded, sequence of DNA with a single-stranded overhang, tethered said pre-defined, double-stranded, sequence of DNA with a single-stranded overhang with a bead, and lengthening said pre-defined, double-stranded, sequences of DNA by the addition of user-selected, single stranded DNA sequences.

8. The method of writing and/or reading information using DNA of claim 1 including the step of decoding said information from said at least one information containing DNA sequence.

9. A method of writing information using DNA, comprising the steps of:
translating said information into information containing DNA sequence,
preselecting a basic DNA sequence,
synthesizing a DNA molecule of user-defined sequence that contains said information containing DNA sequence and said basic DNA sequence.

10. The method of writing information using DNA of claim 9 wherein said step of translating said information into an information containing DNA sequence comprises using a computer program to translate said information.

11. The method of writing information using DNA of claim 9 wherein said step of preselecting a basic DNA sequence comprises using a computer program to preselect basic DNA sequence.

12. The method of writing information using DNA of claim 9 wherein said steps of translating said information into an information containing DNA sequence and preselecting a basic DNA sequence comprise using computational techniques to break said sequences into fragments of defined size and said step of synthesizing a DNA molecule of user-defined sequence comprises assembling said fragments into said DNA molecule of user-defined sequence.

13. The method of writing information using DNA of claim 9 wherein pre-defined strings of DNA bases are located before and after said information containing DNA sequence in said DNA molecule of user-defined sequence.

14. The method of writing information using DNA of claim 9 wherein said steps of synthesizing a DNA molecule of user-defined sequence comprises providing a pre-defined, double-stranded, sequence of DNA with a single-stranded overhang, tethered said pre-defined, double-stranded, sequence of DNA with a single-stranded overhang, and lengthening said pre-defined, double-stranded, sequences of DNA by the addition of user-selected, single stranded DNA sequences.

15. The method of writing information using DNA of claim 9 wherein said steps of synthesizing a DNA molecule of user-defined sequence comprises providing a pre-defined, double-stranded, sequence of DNA with a single-stranded overhang, tethered said pre-defined, double-stranded, sequence of DNA with a single-stranded overhang with a bead, and lengthening said pre-defined, double-stranded, sequences of DNA by the addition of user-selected, single stranded DNA sequences.
